(19) United States
(12) Patent Application Publication
(10) Pub. No.: US 2024/0207456 A1
(43) Pub. Date: Jun. 27, 2024

(54) PHOTOCURABLE COMPOSITIONS AND METHODS FOR DELIVERING RADIATION TO A SUBJECT

(71) Applicant: THERAGENICS CORPORATION, Buford, GA (US)

(72) Inventors: Richard Brauckman, Cumming, GA (US); Jacob Freeman, Gainesville, GA (US)

(21) Appl. No.: 18/335,342

(22) Filed: Jun. 15, 2023

Related U.S. Application Data
(60) Provisional application No. 63/352,714, filed on Jun. 16, 2022.

(51) Int. Cl. A61K 51/06; A61K 45/06; A61K 51/12; C08F 2/50; C08F 222/10

(52) U.S. Cl. CPC A61K 51/06; A61K 45/06; A61K 51/1213; C08F 2/50; C08F 222/1065; A61K 2121/00

(57) ABSTRACT

The disclosure is directed in part to polymerizable compositions that can incorporate a radioactive source and be used to treat subjects with certain conditions, and methods of producing and use thereof.

### CROSS-REFERENCE TO RELATED APPLICATIONS

The present application claims priority to U.S. Provisional Application No. 63/352,714, filed Jun. 16, 2022, which is hereby incorporated by reference in its entirety.

### FIELD

The present embodiments generally relate to polymerizable compositions that can incorporate a radioactive source and used to treat subjects with certain conditions. The present embodiments also relate to methods of forming a 3-dimensional printed article that incorporates the radioactive material that matches the area to be treated.

### BACKGROUND

Radioactive material or seeds are often used to treat various conditions, including skin conditions, such as various types of skin cancer. It has previously been difficult to apply the radioactive source material to a specific area without radiating healthy tissue. Accordingly, there is a need for polymerizable materials that can incorporate radioactive materials and be produced in a manner that matches the area to be treated. The present embodiments fulfills these needs as well as others.

### SUMMARY

The present disclosure provides, in part, a curable composition comprising a first composition comprising one or more monomers, a second composition comprising one or more oligomers, a third composition capable of creating free radicals when exposed to radiation (e.g., ultraviolet, infrared, and the like); and a fourth composition comprising one or more radioisotopes.

In some embodiments, the first composition comprises a styrene, a N-vinylpyrrolidone, an acrylate, or a mixture thereof.

In some embodiments, the second composition comprises an epoxide, a urethanes, a polyethers, a polyester, or any combination thereof.

In some embodiments, the composition comprising one or more radioisotopes is in the form of a solution, colloid, microspheres, or nanoparticles.

In some embodiments, the isotope or isotopes is Pd-103, Pd-109, I-125, I-124, I-123, 1-131, P-32, Y-90, Ac-225, Cs-131, B-10, Ir-192, Sn-177m, Ho-166, Cu-64, Cu-67, Re-186, Re-188, Ga-67, Ga-68, In-111, Co-60, Cs-137, Lu-177, Yb-169, Er-169, Au-198, Sm-153, Am-241, Sr-89, Ra-223, Pb-212, Bi-213, Tc-99, At-211, Cf-252, or any combination thereof.

In some embodiments, the styrene is 3-vinylphenylboronic acid, 1-vinylnaphthalene, 4-vinylbenzylamine, vinyltoluene monomer, 2,2′-(2-vinylanthracene-9,10-diylidene)bis(1,3-dithiole), 4-tert-butylstyrene, 2,3,4,5,6-pentafluorostyrene, 3-aminostyrene, 3-bromostyrene, 2-vinylanthraquinone, 4-fluoro-a-methylstyrene, 4-chlorostyrene, 2-chlorostyrene, chloromethylstyrene, 9-vinylanthracene, sodium p-styrenesulfonate hydrate, 4-methoxystyrene, 2-bromostyrene, 4-bromo-β,β-difluorostyrene, 3-fluorostyrene, 4-n-octylstyrene, 2,2′-(2-vinylanthracene-9,10-diylidene)dimalononitrile, divinylbenzene, 4-(chloromethyl)styrene, 2-vinylphenyl acetate, 3-chlorostyrene, vinylbenzyl cyanide, 3-(trifluoromethyl)styrene, 2,4,6-trimethylstyrene, trimethoxy(4-vinylphenyl)silane, 2-methylstyrene, 4-methylstyrene, 4-vinylbiphenyl, 4-isopropenyltoluene, 4-tert-butoxystyrene, 1-(1-ethoxyethoxy)-4-vinylbenzene, trimethyl(4-vinylphenyl)silane, 4-vinylphenyl acetate, a-methylstyrene, styrene, 4-aminostyrene, 4-fluorostyrene, 4-(trifluoromethyl)styrene, diphenyl(4-vinylphenyl)phosphine, 4-vinylbenzoic acid, 4-vinylphenylboronic acid, 4-nitrostyrene, 4-bromostyrene, 3-methylstyrene, or any combination thereof.

In some embodiments, the isotope is Pd-103.

In some embodiments, the Pd-103 isotope is produced from Pd-102 or Pd-104.

In some embodiments, the isotope is Y-90.

In some embodiments, the isotope is Sn-177m.

In some embodiments, a method of preparing a substantially cured composition, is provided. In some embodiments, the method comprises mixing a curable composition, such as those provided herein, and a photoinitiator to form a substantially cured composition.

In some embodiments, a method of preparing a substantially cured composition, is provided. In some embodiments, the method comprises mixing a curable composition, such as those provided herein, and printing the composition with a 3-D printer in a target area configuration and curing the composition with a photoinitiator to form a substantially cured composition in the shape of the target area configuration.

In some embodiments, a method of treating a subject with radiation, is provided. In some embodiments, the method comprises applying, implanting, contacting, and the like, the cured composition prepared from of any one of those provided herein.

### DETAILED DESCRIPTION

A, An, The: As used herein, the singular forms “a,” “an” and “the” include plural referents unless the context clearly dictates otherwise.

About, Approximately: As used herein, the terms “about” and “approximately” shall generally mean an acceptable degree of error for the quantity measured given the nature or precision of the measurements. Exemplary degrees of error are within 15 percent (%), typically, within 10%, and more typically, within 5% of a given value or range of values.

A “polymer” is a substance composed of macromolecules. A polymer macromolecule is a molecule of high relative molecular mass, the structure of which comprises the multiple repetition of units derived from molecules of low relative molecular mass.

A “branched polymer” is a polymer that includes side chains of repeat units connecting onto the main chain of repeat units (different from side chains already present in the monomers). A branched polymer refers to a non-linear polymer structure, but typically, not a network structure. Therefore, a trace forward from the branch point would not bridge back to the original main chain; i.e. minimal to no backbone crosslinking is present. A branched polymer would generally be soluble in an appropriate solvent.

A “crosslinked polymer” is a polymer that includes interconnections between chains, either formed during polymerization (by choice of monomer) or after polymerization (by addition of a specific reagent). In a crosslinked polymer network, with the crosslinks serving as branch points, it is possible to trace a continuous loop back to the backbone. The crosslinked network would be insoluble in all solvents.

A “network polymer” is a crosslinked polymer that includes two or more connections, on average, between chains such that the entire sample is, or could be, a single molecule. Limited crosslink connections per chain would be considered lightly crosslinked while numerous crosslinks would be considered highly (or heavily) crosslinked.

A “copolymer” is a material created by polymerizing a mixture of two, or more, starting compounds. The resultant polymer molecules contain the monomers in a proportion which is related both to the mole fraction of the monomers in the starting mixture and to the reaction mechanism.

As used herein, the term “subject,” “individual,” or “patient,” used interchangeably, means any animal, including mammals, such as mice, rats, other rodents, rabbits, dogs, cats, swine, cattle, sheep, horses, or primates, such as humans.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent. For example, in some embodiments, the subject is a subject in need of radiation treatment due to a condition such as skin cancer.

As used herein, the phrase “integer from X to Y” means any integer that includes the endpoints. For example, the phrase “integer from 1 to 5” means 1, 2, 3, 4, or 5.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the phrase “therapeutically effective amount” means the amount of active compound, radioisotope, or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, elimination of a disorder, or side-effects. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” mean both therapeutic treatment and prophylactic measures wherein the object is to ameliorate slow down (lessen) an undesired physiological condition, disorder or disease, or obtain beneficial or desired clinical results. The beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects, such as avoiding significantly exposing healthy tissue to radiation. Treatment also includes prolonging survival as compared to expected survival if not receiving treatment.

As used herein, the term “contacting” means bringing together of two elements in an in vitro system or an in vivo system, such as applying or adhering a composition provided for herein to the skin of a subject or implanting the composition into a cavity, organ, tissue, and the like within the subject.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “cancer” means a spectrum of pathological symptoms associated with the initiation or progression, as well as metastasis, of malignant tumors. Examples include, but are not limited to skin cancer. Examples of skin cancer include, but are not limited to, malignant melanoma (MM), basal cell carcinoma (BCC) and squamous cell carcinomas (SCC).

As used herein, the term “animal” includes, but is not limited to, humans and non-human vertebrates such as wild, domestic, and farm animals.

As used herein, the term “about” means that the numerical value is approximate and small variations would not significantly affect the practice of the disclosed embodiments. Where a numerical limitation is used, unless indicated otherwise by the context, “about” means the numerical value can vary by 10% and remain within the scope of the disclosed embodiments. In some embodiments, the numerical value can vary by 5% and remain within the scope of the disclosed embodiments.

In some embodiments, a curable composition is provided that can be used, for example, to treat a condition in a subject that requires radiation to be delivered to a subject, such as a specific tissue or other area on or within the subject. The composition can be a polymeric composition that can be formed from a combination of a polymer, network polymer, crosslinked polymer, a copolymer, a branched polymer and the like.

In some embodiments, the curable composition comprises a first composition comprising one or more monomers, a second composition comprising one or more oligomers, a third composition capable of creating free radicals; and a fourth composition comprising one or more radioisotopes. Without being bound to any theory, the third composition facilitates the polymerization, curing of the first and second composition when the free radicals are generated. The free radicals can be generated via any known method, and as such the third composition may comprise any such components necessary to generate the free radicals. In some embodiments, the free radicals are generated through a chemical reaction taking place within the third composition. In some embodiments, the free radicals are generated when the third composition is exposed to radiation. The radiation can be ultraviolet, infrared, thermal, or any other form of radiation that can be used to facilitate the curing/polymerization of the composition. In some embodiments, the radiation is ultraviolet radiation. In some embodiments, the radiation is infrared radiation. In some embodiments, the radiation is thermal radiation.

In some embodiments, the composition comprises a therapeutically effective amount of the radioisotope. In some embodiments, the radioisotope is present in an amount sufficient to be detected and located. The detection and the location can be by any means known in the art. As a non-limiting example, the radioisotope can be detected with a handheld frisker or Geiger counter and thus the radioisotope located by the readout of the frisker or Geiger counter. As another non-limiting example, the radioisotope can be detected by an image processer and the location rendered by said image processor. Accordingly, in some embodiments, the radioisotope is present in an amount sufficient to be detected and located by a handheld frisker, such as a Geiger counter. In some embodiments, the radioisotope is present in an amount sufficient to be detected and located by an image processor. In some embodiments, the radioisotope is present in an amount sufficient to deliver a therapeutic dose of radiation to a mammal.

In some embodiments, the one or more radioisotopes are therapeutic radioisotopes, diagnostic radioisotopes, or a combination thereof. As used herein, a “therapeutic radioisotope” is any radioisotope that may be used to deliver therapeutic radiation to a subject in need thereof for the treatment of a disease or disorder. As used herein, a “diagnostic radioisotope” is any radioisotope that may be used is combination with an imaging unit and an image processor to assist in the diagnosis of any disease or disorder. It is to be understood that “therapeutic radioisotope” and “diagnostic radioisotope” are not mutually exclusive.

In some embodiments, the one or more radioisotopes comprise a diagnostic radioisotope. In some embodiments the diagnostic radioisotope is present in an amount sufficient to be detected and located. The detection and the location can be by any means known in the art. As a non-limiting example, the radioisotope can be detected with a handheld frisker or Geiger counter and thus the radioisotope located by the readout of the frisker or Geiger counter. As another non-limiting example, the radioisotope can be detected by an image processer and the location rendered by said image processor. Accordingly, in some embodiments, the radioisotope is present in an amount sufficient to be detected and located by a handheld frisker, such as a Geiger counter. In some embodiments, the radioisotope is present in an amount sufficient to be detected and located by an image processor. In some embodiments, the diagnostic radioisotope is produced from one or more radioisotope precursors. In some embodiments, the radioisotope precursors are present in an amount sufficient to produce a diagnostic radioisotope in an amount sufficient to be detected and located as provided for herein.

In some embodiments, the one or more radioisotope comprises a therapeutic radioisotope. In some embodiments, the therapeutic radioisotope is present in an amount sufficient to deliver a therapeutic dose of radiation to a mammal. In some embodiments, the therapeutic radioisotope is produced from one or more radioisotope precursors. In some embodiments, the radioisotope precursors are present in an amount sufficient to produce a therapeutic radioisotope in an amount sufficient to deliver a therapeutic dose of radiation to a mammal.

In some embodiments, the one or more radioisotopes are uniformly distributed throughout the curable composition. In some embodiments, the one or more radioisotopes are not uniformly distributed throughout the curable composition. Without being bound to any particular theory, a radioisotope of the present disclosure could be spatially distributed throughout the curable composition such that a portion or portions of the composition comprise a higher regional concentration of radioisotopes than other regions of the composition. Such a composition would be useful, for example, to generate radiation dosimetry that conforms to a particular treatment area.

In some embodiments, the curable composition is biocompatible. As used herein, the term “biocompatible” refers to a composition that can be used with a subject, such as a human or animal without causing significant adverse or negative reactions to the composition.

In some embodiments, the curable composition is bioabsorbable. A “bioabsorbable” composition is one that is absorbed or degraded over time.

In some embodiments, the one or more monomers of the first composition comprises a styrene, a N-vinylpyrrolidone, an acrylate, or a mixture thereof. In some embodiments, the one or more monomers of the first composition comprises a styrene. In some embodiments, the one or more monomers of the first composition comprises a N-vinylpyrrolidone. In some embodiments, the one or more monomers of the first composition comprises an acrylate. In some embodiments, the one or more monomers of the first composition comprises a combination of a styrene and a N-vinylpyrrolidone. In some embodiments, the one or more monomers of the first composition comprises a combination of a styrene and an acrylate. In some embodiments, the one or more monomers of the first composition comprises a combination of a N-vinylpyrrolidone and an acrylate. In some embodiments, the one or more monomers of the first composition comprises a combination of a styrene, a N-vinylpyrrolidone, and an acrylate.

The styrene of the first composition may be any suitable styrene known in the art. Examples of a “styrene,” include, but are not limited to is 3-vinylphenylboronic acid, 1-vinylnaphthalene, 4-vinylbenzylamine, vinyltoluene monomer, 2,2′-(2-vinylanthracene-9,10-diylidene)bis(1,3-dithiole), 4-tert-butylstyrene, 2,3,4,5,6-pentafluorostyrene, 3-aminostyrene, 3-bromostyrene, 2-vinylanthraquinone, 4-fluoro-α-methylstyrene, 4-chlorostyrene, 2-chlorostyrene, chloromethylstyrene, 9-vinylanthracene, sodium p-styrenesulfonate hydrate, 4-methoxystyrene, 2-bromostyrene, 4-bromo-β,β-difluorostyrene, 3-fluorostyrene, 4-n-octylstyrene, 2,2′-(2-vinylanthracene-9,10-diylidene)dimalononitrile, divinylbenzene, 4-(chloromethyl)styrene, 2-vinylphenyl acetate, 3-chlorostyrene, vinylbenzyl cyanide, 3-(trifluoromethyl)styrene, 2,4,6-trimethylstyrene, trimethoxy(4-vinylphenyl)silane, 2-methylstyrene, 4-methylstyrene, 4-vinylbiphenyl, 4-isopropenyltoluene, 4-tert-butoxystyrene, 1-(1-ethoxyethoxy)-4-vinylbenzene, trimethyl(4-vinylphenyl)silane, 4-vinylphenyl acetate, α-methylstyrene, styrene, 4-aminostyrene, 4-fluorostyrene, 4-(trifluoromethyl)styrene, diphenyl(4-vinylphenyl)phosphine, 4-vinylbenzoic acid, 4-vinylphenylboronic acid, 4-nitrostyrene, 4-bromostyrene, 3-methylstyrene, or any combination thereof. In some embodiments, the styrene is 3-vinylphenylboronic acid. In some embodiments, the styrene is 1-vinylnaphthalene. In some embodiments, the styrene is 4-vinylbenzylamine. In some embodiments, the styrene is vinyltoluene monomer. In some embodiments, the styrene is 2,2′-(2-vinylanthracene-9,10-diylidene)bis(1,3-dithiole). In some embodiments, the styrene is 4-tert-butylstyrene, 2,3,4,5,6-pentafluorostyrene. In some embodiments, the styrene is 3-aminostyrene. In some embodiments, the styrene is 3-bromostyrene. In some embodiments, the styrene is 2-vinylanthraquinone. In some embodiments, the styrene is 4-fluoro-α-methylstyrene. In some embodiments, the styrene is 4-chlorostyrene. In some embodiments, the styrene is 2-chlorostyrene. In some embodiments, the styrene is chloromethylstyrene. In some embodiments, the styrene is 9-vinylanthracene. In some embodiments, the styrene is sodium p-styrenesulfonate hydrate. In some embodiments, the styrene is 4-methoxystyrene. In some embodiments, the styrene is 2-bromostyrene. In some embodiments, the styrene is 4-bromo-β,β-difluorostyrene. In some embodiments, the styrene is 3-fluorostyrene. In some embodiments, the styrene is 4-n-octylstyrene. In some embodiments, the styrene is 2,2′-(2-vinylanthracene-9,10-diylidene)dimalononitrile. In some embodiments, the styrene is divinylbenzene. In some embodiments, the styrene is 4-(chloromethyl)styrene. In some embodiments, the styrene is 2-vinylphenyl acetate. In some embodiments, the styrene is 3-chlorostyrene. In some embodiments, the styrene is vinylbenzyl cyanide. In some embodiments, the styrene is 3-(trifluoromethyl)styrene. In some embodiments, the styrene is 2,4,6-trimethylstyrene. In some embodiments, the styrene is trimethoxy(4-vinylphenyl)silane. In some embodiments, the styrene is 2-methylstyrene. In some embodiments, the styrene is 4-methylstyrene. In some embodiments, the styrene is 4-vinylbiphenyl. In some embodiments, the styrene is 4-isopropenyltoluene. In some embodiments, the styrene is 4-tert-butoxystyrene. In some embodiments, the styrene is 1-(1-ethoxyethoxy)-4-vinylbenzene. In some embodiments, the styrene is trimethyl(4-vinylphenyl)silane. In some embodiments, the styrene is 4-vinylphenyl acetate. In some embodiments, the styrene is α-methylstyrene. In some embodiments, the styrene is styrene. In some embodiments, the styrene is 4-aminostyrene. In some embodiments, the styrene is 4-fluorostyrene. In some embodiments, the styrene is 4-(trifluoromethyl)styrene. In some embodiments, the styrene is diphenyl(4-vinylphenyl)phosphine. In some embodiments, the styrene is 4-vinylbenzoic acid. In some embodiments, the styrene is 4-vinylphenylboronic acid.

In some embodiments, the styrene is 4-nitrostyrene. In some embodiments, the styrene is 4-bromostyrene. In some embodiments, the styrene is 3-methylstyrene. In some embodiments, the styrene is any combination of any of the foregoing.

The acrylate of the first composition may be any suitable acrylate known in the art. Examples of acrylates that can be used, include, but are not limited to, t-butyl acrylate, t-butyl acrylamide, n-octyl methacrylate, methyl methacrylate, hydroxyethyl methacrylate, hydroxyethyl acrylate, hydroxypropyl methacrylate, hydroxybutyl methacrylate, derivatives thereof and combinations thereof. In some embodiments, the acrylate is t-butyl acrylate, or a derivative thereof. In some embodiments, the acrylate is t-butyl acrylamide, or a derivative thereof. In some embodiments, the acrylate is n-octyl methacrylate, or a derivative thereof. In some embodiments, the acrylate is methyl methacrylate, or a derivative thereof. In some embodiments, the acrylate is hydroxyethyl methacrylate, or a derivative thereof. In some embodiments, the acrylate is hydroxyethyl acrylate, or a derivative thereof. In some embodiments, the acrylate is hydroxypropyl methacrylate, or a derivative thereof. In some embodiments, the acrylate is hydroxybutyl methacrylate, or a derivative thereof. In some embodiments, the acrylate is a combination of any of the foregoing. In some embodiments, the acrylate is hydroxyethyl methacrylate (HEMA).

In some embodiments the oligomers of the second composition comprise an epoxide (epoxide-containing compound), urethanes, polyethers, a polyester, or any combination thereof. In some embodiments, the oligomers of the second composition comprise an epoxide (epoxide-containing compound). In some embodiments, the oligomers of the second composition comprise urethanes. In some embodiments, the oligomers of the second composition comprise polyethers. In some embodiments, the oligomers of the second composition comprise a polyester. In some embodiments, the oligomers of the second composition comprise a combination of an epoxide and urethanes. In some embodiments, the oligomers of the second composition comprise a combination of an epoxide and polyetheres. In some embodiments, the oligomers of the second composition comprise a combination of an epoxide and a polyester. In some embodiments, the oligomers of the second composition comprise a combination of urethanes and polyethers. In some embodiments, the oligomers of the second composition comprise a combination of urethanes and a polyester. In some embodiments, the oligomers of the second composition comprise a combination an epoxide, urethanes, and polyethers. In some embodiments, the oligomers of the second composition comprise a combination an epoxide, urethanes, and a polyester. In some embodiments, the oligomers of the second composition comprise a combination an epoxide, polyethers, and a polyester. In some embodiments, the oligomers of the second composition comprise a combination an epoxide, urethanes, polyethers, and a polyester.

The term “epoxide”, “epoxy group” refers to a chemical functional group consisting of a three-membered ring arrangement of two carbon atoms and one oxygen atom. The two carbon atoms in the three-membered ring may be independently substituted. The term “epoxide” may also depict a molecule or compound that comprises at least one epoxy group. The term “epoxide-containing compound” refers to any compound that is an epoxide or a compound which contains an epoxide moiety. Non-limiting epoxide containing compounds are alkylene oxides, such as lower alkylene oxides such as ethylene oxide, propylene oxide, butylene oxide, alcohol epoxides such as glycidol, and epihalohydrins such as epichlorohydrin, epibromohydrin, epiiodohydrin, 1,2-epoxy-4-chlorobutane, 1,2-epoxy-4-bromobutane, 1,2-epoxy-4-iodobutane, 2,3-epoxy-4-chlorobutane, 2,3-epoxy-4-bromobutane, 2,3-epoxy-4-iodobutane, 2,3-epoxy-5-chloropentane, 2,3-epoxy-5-bromopentane, 1,2-epoxy-5-chloropentane, etc., epoxy compounds such as 2,2-bis(p-1,2-epoxypropoxyphenyl)-propane, 1,4-bis(1,2-epoxypropoxy)benzene, N,N′-bis(2,3-epoxypropyl)piperazine, and the like. In some embodiments, the epoxide containing compounds are alkylene oxides. In some embodiments, the epoxide containing compound are alkylene oxides. In some embodiments, the epoxide containing compound is ethylene oxide. In some embodiments, the epoxide containing compound is propylene oxide. In some embodiments, the epoxide containing compound is butylene oxide. In some embodiments, the epoxide containing compound are alcohol epoxides. In some embodiments, the epoxide containing compound is glycidol. In some embodiments, the epoxide containing compound is an epihalohydrin. In some embodiments, the epoxide containing compound is epichlorohydrin. In some embodiments, the epoxide containing compound is epibromohydrin. In some embodiments, the epoxide containing compound is epiiodohydrin. In some embodiments, the epoxide containing compound is 1,2-epoxy-4-chlorobutane. In some embodiments, the epoxide containing compound is 1,2-epoxy-4-bromobutane. In some embodiments, the epoxide containing compound is 1,2-epoxy-4-iodobutane. In some embodiments, the epoxide containing compound is 2,3-epoxy-4-chlorobutane. In some embodiments, the epoxide containing compound is 2,3-epoxy-4-bromobutane. In some embodiments, the epoxide containing compound is 2,3-epoxy-4-iodobutane. In some embodiments, the epoxide containing compound is 2,3-epoxy-5-chloropentane. In some embodiments, the epoxide containing compound is 2,3-epoxy-5-bromopentane. In some embodiments, the epoxide containing compound is 1,2-epoxy-5-chloropentane. In some embodiments, the epoxide containing compound is 2,2-bis(p-1,2-epoxypropoxyphenyl)-propane. In some embodiments, the epoxide containing compound is 1,4-bis(1,2-epoxypropoxy)benzene. In some embodiments, the epoxide containing compound is N,N′-bis(2,3-epoxypropyl)piperazine. In some embodiments, the epoxide containing compounds are a combination of any of the forgoing.

In some embodiments, the urethane is any suitable urethane. In some embodiments, the urethane is a polyurethane oligomer.

In some embodiments, the polyether is any suitable polyether. In some embodiments, the polyether is polyethylene oxide or polypropylene oxide. In some embodiments, the polyether is polyethylene oxide. In some embodiments, the polyether is polypropylene oxide.

In some embodiments, the polyester is any suitable polyester. In some embodiments, the polyester is polylactic acid (PLA), polyglycolic acid, PGA or a co-polymer of polylactic acid and polyglycolic acid (PLGA). In some embodiments, the polyester is PLA. In some embodiments, the polyester is PGA. In some embodiments, the polyester is PLGA.

The composition comprising one or more radioisotopes can be in any form, such as, but not limited to, a solution, colloid, microspheres, or nanoparticles. In some embodiments, the fourth composition comprising one or more radioisotopes can be in any form, such as, but not limited to, a solution, colloid, microspheres, or nanoparticles. In some embodiments, the fourth composition is in the form of a solution. In some embodiments, the fourth composition is in the form of a colloid. In some embodiments, the fourth composition is in the form of microspheres. In some embodiments, the fourth composition is in the form of nanoparticles.

In some embodiments, the one or more radioisotopes comprises Pd-103, I-125, P-32, Y-90, Ac-225, Cs-131, B-10, Ir-192, Sn-177m, Ho-166, Pd-109, Cu-64, Cu-67, Re-186, Re-188, Ga-67, Ga-68, In-111, Co-60, Cs-137, Lu-177, Yb-169, Er-169, Au-198, Sm-153, Am-241, Sr-89, Ra-223, Pb-212, Bi-213, Tc-99, At-211, Cf-252, or any combination thereof. In some embodiments, the radioisotope is Pd-103. In some embodiments, the radioisotope is I-125. In some embodiments, the radioisotope is P-32. In some embodiments, the radioisotope is Y-90. In some embodiments, the radioisotope is Ac-225. In some embodiments, the radioisotope is Cs-131. In some embodiments, the radioisotope is B-10. In some embodiments, the radioisotope is Ir-192. In some embodiments, the radioisotope is Sn-177m. In some embodiments, the radioisotope is Ho-166. In some embodiments, the radioisotope is Pd-109. In some embodiments, the radioisotope is Cu-64. In some embodiments, the radioisotope is Cu-67. In some embodiments, the radioisotope is Re-186. In some embodiments, the radioisotope is Re-188. In some embodiments, the radioisotope is Ga-67. In some embodiments, the radioisotope is Ga-68. In some embodiments, the radioisotope is In-111. In some embodiments, the radioisotope is Co-60. In some embodiments, the radioisotope is Cs-137. In some embodiments, the radioisotope is Lu-177. In some embodiments, the radioisotope is Yb-169. In some embodiments, the radioisotope is Er-169. In some embodiments, the radioisotope is Au-198. In some embodiments, the radioisotope is Sm-153. In some embodiments, the radioisotope is Am-241. In some embodiments, the radioisotope is Sr-89. In some embodiments, the radioisotope is Ra-223. In some embodiments, the radioisotope is Pb-212. In some embodiments, the radioisotope is Bi-213. In some embodiments, the radioisotope is Tc-99. In some embodiments, the radioisotope is At-211. In some embodiments, the radioisotope is Cf-252. In some embodiments, the fourth composition comprises a combination of radioisotopes, wherein the radioisotopes are selected from any of the foregoing. In some embodiments, the radioisotope is Pd-103. In some embodiments, the Pd-103 is produced from Pd-102, or Pd-104. In some embodiments, the radioisotope is Y-90. In some embodiments, the Y-90 is produced from Y-89 or Sr-90.

In some embodiments, the one or more radioisotopes in the fourth composition is produced by activating one or more radioisotope precursors. As used herein, “activating” or “activation” or the one or more radioisotope precursors refers to any process or method known in the art to produce a desired radioisotope from a give radioisotope precursor. For example, activating the radioisotope precursor can involve bombarding (exposing) the precursor with energetic particles. The bombarding particles include, but are not limited to, alpha particles (a), neutrons (n), ³He atoms, protons (p), tritons (t), gamma rays (γ), heavy ions, and deuterons (d). Typical sources of these types of bombardments are nuclear reactors and particle accelerators. Examples of precursor activation include, but are not limited to: using a (p,n) reaction to produce ¹⁰³pd: ¹⁰³Rh (p,n)→¹⁰³Pd where “p” is a proton in and “n” is a neutron out; using a (n,7) reaction to produce ¹⁰³Pd: ¹⁰²Pd (n,7)→¹⁰³Pd where “n” is a neutron in and “7” is a gamma ray out; using a (p,2n) reaction to produce ¹⁰³Pd: ¹⁰⁴Pd (p,2n)→¹⁰³Ag (ε)→¹⁰³Pd where “p” is a proton in and “2n” is 2 neutrons out, “C” is electron capture; using a (n,7) reaction to produce ³²P: ³¹P (n,T)→³²P where “n” is a neutron in and “γ” is a gamma ray out; using a (n,a) reaction to produce ⁷Li and an α: 1° B (n,a)→⁷Li where “n” is a neutron in and “α” is an alpha out; or using a (n,γ) reaction to produce ⁹⁰Y: ⁸⁹Y (n,Γ)γ⁹⁰Y where “n” is a neutron in and “y” is a gamma ray out. Additional reactions for precursor activation include but are not limited to (α,3n), (α,2n), (α,n), (p,γ), (d,n), (³He,np), (α,np), (t,n), (³He,p), (p,pn), (γ,n), (n,2n), (d,p), (t,np), (t,p), (p,α), (n,t), (γ,np), (n,nd), (n,d), (γ,p), (n,np), (n,p), (t, ³He), (n,a), (n,n³He), (n,³He), (p,3n), (p,4n), and (n,pd). In some embodiments, this can be utilized, by activating a precursor in a cured composition. For example, in some embodiments, an isotopically engineered precursor is incorporated into a curable device that is then cured. The cured device can then exposed to a neutron beam of sufficient flux for a sufficient period of time to activate the precursor to a predetermined level of activity. This activity can be determined the person that is sufficient for the purpose. In some embodiments, the activated device can then be applied to a patient to deliver a therapeutic dose of the radiological material to the region it is designed to treat. In some embodiments, another isotopically engineered precursor can be used and use the same or similar process, but instead of a neutron beam the device is exposed to proton beam of sufficient energy and current to activate the precursor to a predetermined level of activity and then is used to treat a patient as described herein.

The radioisotope precursor may be any precursor necessary or useful to produce a radioisotope as provided for herein. Non-limiting examples of radioisotope precursors include, but are not limited to, Xe-124, S-32, Y-89, Sr-90, Th-229, Ir-191, Ir-193, Dy-164, Pd-102, Pd-104, Pd-108, B-10, P-31, Ni-64, Zn-67, Zn-68, Zn-70, Rh-103, Te-125, Te-124, Ba-130, Cs-133, Th-232, Sr-86, Er-168, Cd-111, Cd-112, W-186, Bi-209, Tm-169, Yb-176, Ra-226, or any combination thereof. In some embodiments, the radioisotope precursor is Xe-124. In some embodiments, the radioisotope precursor is S-32. In some embodiments, the radioisotope precursor is Y-89. In some embodiments, the radioisotope precursor is Sr-90. In some embodiments, the radioisotope precursor is Th-229. In some embodiments, the radioisotope precursor is Ir-191. In some embodiments, the radioisotope precursor is Ir-193. In some embodiments, the radioisotope precursor is Dy-164. In some embodiments, the radioisotope precursor is Pd-102. In some embodiments, the radioisotope precursor is Pd-104. In some embodiments, the radioisotope precursor is Pd-108. In some embodiments, the radioisotope precursor is B-10. In some embodiments, the radioisotope precursor is P-31. In some embodiments, the radioisotope precursor is Ni-64. In some embodiments, the radioisotope precursor is Zn-67. In some embodiments, the radioisotope precursor is Zn-68. In some embodiments, the radioisotope precursor is Zn-70. In some embodiments, the radioisotope precursor is Rh-103. In some embodiments, the radioisotope precursor is Te-125. In some embodiments, the radioisotope precursor is Te-124. In some embodiments, the radioisotope precursor is Th-232. In some embodiments, the radioisotope precursor is Sr-86. In some embodiments, the radioisotope precursor is Er-168. In some embodiments, the radioisotope precursor is Cd-111. In some embodiments, the radioisotope precursor is Cd-112. In some embodiments, the radioisotope precursor is W-186. In some embodiments, the radioisotope precursor is Bi-209. In some embodiments, the radioisotope precursor is Tm-169. In some embodiments, the radioisotope precursor is Yb-176. In some embodiments, the radioisotope precursor is Ra-226. In some embodiments, the fourth composition comprises a combination of radioisotope precursors, wherein the radioisotope precursors are selected from any of the foregoing. In some embodiments, the radioisotope precursor is Pd-102 and the radioisotope produced is Pd-103. In some embodiments, the radioisotope precursor is Pd-104 and the radioisotope produced is Pd-103. In some embodiments, the radioisotope precursor is Rh-103 and the radioisotope produced is Pd-103. In some embodiments, the radioisotope precursor is Pd-108 and the radioisotope produced is Pd-109. In some embodiments, the radioisotope precursor is Xe-124 and the radioisotope produced is I-125. In some embodiments, the radioisotope precursor is S-32 and the radioisotope produced is P-32. In some embodiments, the radioisotope precursor is P-31 and the radioisotope produced is P-32. In some embodiments, the radioisotope precursor is Y-89 and the radioisotope produced is Y-90. In some embodiments, the radioisotope precursor is Sr-90 and the radioisotope produced is Y-90. In some embodiments, the radioisotope precursor is Th-229 and the radioisotope produced is Ac-225. In some embodiments, the radioisotope precursor is Ba-130 and the radioisotope produced is Cs-131. In some embodiments, the radioisotope precursor is Ir-191 and the radioisotope produced is Ir-192. In some embodiments, the radioisotope precursor is Ir-193 and the radioisotope produced is Ir-192. In some embodiments, the radioisotope precursor is Dy-164 and the radioisotope produced is Ho-166. In some embodiments, the radioisotope precursor is Ni-64 and the radioisotope produced is Cu-64.

In some embodiments, the radioisotope precursor is Zn-67 and the radioisotope produced is Cu-67. In some embodiments, the radioisotope precursor is Zn-68 and the radioisotope produced is Cu-67. In some embodiments, the radioisotope precursor is Zn-70 and the radioisotope produced is Cu-67. In some embodiments, the radioisotope precursor is Cd-111 and the radioisotope produced is In-111. In some embodiments, the radioisotope precursor is Cd-112 and the radioisotope produced is In-111. In some embodiments, the radioisotope precursor is Tm-169 and the radioisotope produced is Yb-169. In some embodiments, the radioisotope precursor is Yb-176 and the radioisotope produced is Lu-177. In some embodiments, the radioisotope precursor is Ra-226 and the radioisotope produced is Pb-212. It is to be understood that the preceding embodiments are exemplary only and are not limiting in any way. The skilled artisan would readily recognize alternative radioisotope precursors that might generate a radioisotope as provided for herein. Similarly, the skilled artisan would readily recognize alternative radioisotopes that might be generated from the radioisotope precursors provided for herein. Such alternative radioisotope precursors and alternative radioisotopes are withing the scope of the present disclosure.

The radioisotopes or radioisotope precursors as provided for herein may be in any suitable form for inclusion in the fourth composition of the curable composition. As a non-limiting example, the radioisotope Pd-103 may be included in the fourth composition as PdCl₂ or Pd(HNO₃)₂Cl₂, wherein the palladium of PdCl₂ or Pd(HNO₃)₂Cl₂ is Pd-103. Similarly, the radioisotope precursor Pd-102 may be included in the fourth composition as PdCl₂ or Pd(HNO₃)₂Cl₂, wherein the palladium of PdCl₂ or Pd(HNO₃)₂Cl₂ is Pd-102. In some embodiments, the radioisotope is Pd-103 and is provided as PdCl₂. In some embodiments, the radioisotope is Pd-103 and is provided as Pd(HNO₃)₂Cl₂. In some embodiments, the radioisotope precursor is Pd-102 and is provided as PdCl₂. In some embodiments, the radioisotope precursor is Pd-102 and is provided as Pd(HNO₃)₂Cl₂. As another non-limiting example, the radioisotope Y-90 may be included in the fourth composition as Y₂O₃, Y₂S₃, O₁₂S₃Y₂, or YF₃, wherein the yttrium of Y₂O₃, Y₂S₃, O₁₂S₃Y₂, or YF₃Y-90. Similarly, the radioisotope precursor Y-89 may be included in the fourth composition as Y₂O₃, Y₂S₃, O₁₂S₃Y₂, or YF₃, wherein the yttrium of Y₂O₃, Y₂S₃, O₁₂S₃Y₂, or YF₃Y-89. The preceding examples are exemplary only and are not meant to be limiting in any way. The skilled artisan would readily recognize additional forms of the radioisotopes provided for herein that might be used in the present composition. Such forms are within the scope of the present disclosure.

In some embodiments, the one or more radioisotopes of the fourth composition is produced by activating the one or more radioisotope precursors prior to the curable composition being cured. In some embodiments, the one or more radioisotopes of the fourth composition is produced by activating the one or more radioisotope precursors after the curable composition has been cured.

Accordingly, in some embodiments, a curable composition is provided, the composition comprising a first composition comprising one or more monomers, a second composition comprising one or more oligomers, a third composition capable of creating free radicals; and a fourth composition comprising one or more radioisotope precursors. The free radicals can be generated via any known method, and as such the third composition may comprise any such components necessary to generate the free radicals. In some embodiments, the free radicals are generated through a chemical reaction taking place within the third composition. In some embodiments, the free radicals are generated when the third composition is exposed to radiation. The radiation can be ultraviolet, infrared, thermal, or any other form of radiation that can be used to facilitate the curing/polymerization of the composition. In some embodiments, the radiation is ultraviolet radiation. In some embodiments, the radiation is infrared radiation. In some embodiments, the radiation is thermal radiation. In some embodiments, the one or more monomers of the first composition comprises a styrene, a N-vinylpyrrolidone, an acrylate, or a mixture thereof, wherein the styrene is as provided for herein and wherein the acrylate is as provided for herein. In some embodiments, the one or more oligomers of the second composition comprise an epoxide (epoxide-containing compound), urethanes, polyethers, a polyester, or any combination thereof, wherein the epoxide-containing compound is as provided for herein, the urethanes are as provided for herein, the polyethers are as provided for herein, and wherein the polyester is as provided for herein. In some embodiments, the one or more radioisotope precursors is as provided for herein.

In some embodiments, the one or more radioisotope precursors are uniformly distributed throughout the curable composition. In some embodiments, the one or more radioisotope precursors are not uniformly distributed throughout the curable composition. Without being bound to any particular theory, a radioisotope precursor of the present disclosure could be spatially distributed throughout the curable composition such that a portion or portions of the composition comprise a higher regional concentration of radioisotope precursors than other regions of the composition. Such a composition would be useful, for example, to generate radiation dosimetry that conforms to a particular treatment area.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope that is Pd-103. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope that is Y-90. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope that is Sn-177m. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope, wherein the radioisotope is Pd 103 provided as PdCl₂. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope, wherein the radioisotope is Pd 103 provided as Pd(HNO₃)₂Cl₂. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope, wherein the radioisotope is Y-90 produced from Y-89. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope, wherein the radioisotope is Y-90 produced from Sr-90. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope precursor that is Pd-102. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope precursor that is Pd-104. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope precursor that is Y-89. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope precursor that is Sr-90. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope precursor that is Pd-102 provided as PdCl₂. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope precursor that is Pd-102 provided as Pd(HNO₃)₂Cl₂. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope precursor that is Pd-104 provided as PdCl₂. In some embodiments, the methylacrylate is 2-Hydroxyethyl Methacrylate (HEMA). In some embodiments, the acrylate oligomer is urethane acrylate.

In some embodiments, a curable composition is provided, wherein the composition comprises a first composition comprising a monomer that is a methylacrylate, a second composition comprising an oligomer that is an acrylate, a third composition capable of creating free radicals when exposed to radiation, and a fourth composition comprising a radioisotope precursor that is Pd-104 provided as Pd(HNO₃)₂Cl₂. In some embodiments, the methylacrylate is 2-Hydroxyethyl Methacrylate (HEMA). In some embodiments, the acrylate oligomer is urethane acrylate.

In some embodiments, the third composition of the curable composition comprises any necessary components to generate free radicals. In some embodiments, the third composition of the curable composition comprises a photoinitiator, which can be a photochemical. Examples of such initiators, include, but are not limited to tert-Butyl peroxyneodecanoate, benzoyl peroxide, dicumyl peroxide, methyl ethyl ketone peroxide, lauryl peroxide, cyclohexanone peroxide, t-butyl perbenzoate, tbutyl hydroperoxide, t-butylbenzene hydroperoxide, cumene hydroperoxide, t-butyl peroctoate, azobis-isobutyronitrile, 2-tbutylazo-2-cyano-4-methylpentane, and 4-t-butylazo-4-cyano-valeric acid. In some embodiments, the photoinitiator is tert-Butyl peroxyneodecanoate. In some embodiments, the photoinitiator is benzoyl peroxide. In some embodiments, the photoinitiator is dicumyl peroxide. In some embodiments, the photoinitiator is methyl ethyl ketone peroxide. In some embodiments, the photoinitiator is lauryl peroxide. In some embodiments, the photoinitiator is cyclohexanone peroxide. In some embodiments, the photoinitiator is t-butyl perbenzoate. In some embodiments, the photoinitiator is tbutyl hydroperoxide. In some embodiments, the photoinitiator is t-butylbenzene hydroperoxide. In some embodiments, the photoinitiator is cumene hydroperoxide. In some embodiments, the photoinitiator is t-butyl peroctoate. In some embodiments, the photoinitiator is azobis-isobutyronitrile. In some embodiments, the photoinitiator is 2-tbutylazo-2-cyano-4-methylpentane. In some embodiments, the photoinitiator is 4-t-butylazo-4-cyano-valeric acid. In some embodiments, the composition comprises a combination of at least two photoinitiators, wherein the at least two photoinitiators are selected from tert-Butyl peroxyneodecanoate, benzoyl peroxide, dicumyl peroxide, methyl ethyl ketone peroxide, lauryl peroxide, cyclohexanone peroxide, t-butyl perbenzoate, tbutyl hydroperoxide, t-butylbenzene hydroperoxide, cumene hydroperoxide, t-butyl peroctoate, azobis-isobutyronitrile, 2-t-butylazo-2-cyano-4-methylpentane, 4-t-butylazo-4-cyano-valeric acid, and the like, or any combination thereof.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope that is Pd-103. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope that is Y-90. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope that is Sn-177m. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope, wherein the radioisotope is Pd 103 provided as PdCl₂. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope, wherein the radioisotope is Pd 103 provided as Pd(HNO₃)₂Cl₂. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope, wherein the radioisotope is Y-90 produced from Y-89. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope, wherein the radioisotope is Y-90 produced from Sr-90. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope precursor that is Pd-102. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope precursor that is Pd-104. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope precursor that is Y-89. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope precursor that is Sr-90. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope precursor that is Pd-102 provided as PdCl₂. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope precursor that is Pd-102 provided as Pd(HNO₃)₂Cl₂. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope precursor that is Pd-104 provided as PdCl₂. In some embodiments, the methylacrylate is 2-Hydroxyethyl Methacrylate (HEMA). In some embodiments, the acrylate oligomer is urethane acrylate. In some embodiments, the photoinitiator is as provided herein.

In some embodiments, a curable composition is provided, wherein the composition comprises a first composition comprising a monomer that is a methylacrylate, a second composition comprising an oligomer that is an acrylate, a third composition comprising a photoinitiator, and a fourth composition comprising a radioisotope precursor that is Pd-104 provided as Pd(HNO₃)₂Cl₂. In some embodiments, the methylacrylate is 2-Hydroxyethyl Methacrylate (HEMA). In some embodiments, the acrylate oligomer is urethane acrylate. In some embodiments, the photoinitiator is as provided herein.

Also provided herein are substantially cured compositions comprising a polymeric structure prepared from the curable composition as provided for herein. As used herein, “substantially cured” refers to a composition that is at least 50% cured. Accordingly, a substantially cured composition may be at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% cured.

Also provided herein are methods of preparing a substantially cured or cured composition. In some embodiments, the methods comprise mixing a curable composition as provided for herein and mixing the components under conditions sufficient to cure the composition. In some embodiments, the method comprises mixing a curable composition as provided for herein and exposing the curable composition to radiation to form a substantially cured or cured composition. In some embodiments, the third composition of the curable composition comprises a photoinitiator as provided for herein. In some embodiments, the methods comprises exposing the composition to UV light or infrared light to initiate the curing of the composition. In some embodiments, the composition is exposed as a wavelength of about 380-420 nm. In some embodiments, the composition is exposed as a wavelength of about 405 nm.

In some embodiments, a method is provided for preparing a multi-layered substantially cured or cured composition. In some embodiments, the method comprises preparing a first layer of a substantially cured or cured composition, wherein the preparing comprises mixing the curable compositions as provided for herein and exposing the curable composition to radiation to form a first layer of a substantially cured or cured composition. In some embodiments, the method comprises preparing a second layer of a substantially cured or cured composition on top of the first layer, the method comprising mixing a curable composition as provided for herein, placing the mixture on top of the first layer, and exposing the mixture to radiation to form the second layer of a substantially cured or cured composition on top of the first layer. This process can be repeated as many times as necessary for form a multi-layered composition. In some embodiments, the methods are repeated to form a 1, 2, 3, 4, 5, 6, 7, 8, 9 or 10 layer composition. In some embodiments, the substantially cured or cured composition is a 1 layer substantially cured or cured composition. In some embodiments, the substantially cured or cured composition is a 2 layer substantially cured or cured composition. In some embodiments, the substantially cured or cured composition is a 3 layer substantially cured or cured composition. In some embodiments, the substantially cured or cured composition is a 4 layer substantially cured or cured composition. In some embodiments, the substantially cured or cured composition is a 5 layer substantially cured or cured composition. In some embodiments, the substantially cured or cured composition is a 6 layer substantially cured or cured composition. In some embodiments, the substantially cured or cured composition is a 7 layer substantially cured or cured composition. In some embodiments, the substantially cured or cured composition is an 8 layer substantially cured or cured composition. In some embodiments, the substantially cured or cured composition is a 9 layer substantially cured or cured composition. In some embodiments, the substantially cured or cured composition is a 10 layer substantially cured or cured composition. In some embodiments, the substantially cured or cured composition comprises more than 10 layers. In some embodiments, the method is repeated to form a 3 layer cured or substantially cured composition.

Accordingly, provided herein are multi-layer cured or substantially cured compositions that are prepared from the curable compositions provided for herein. In some embodiments, the compositions comprises 1, 2, 3, 4, 5, 6, 7, 8, 9 or 10 layers. In some embodiments, the compositions comprises 1 layers. In some embodiments, the compositions comprises 2 layers. In some embodiments, the compositions comprises 3 layers. In some embodiments, the compositions comprises 4 layers. In some embodiments, the compositions comprises 5 layers. In some embodiments, the compositions comprises 6 layers. In some embodiments, the compositions comprises 7 layers. In some embodiments, the compositions comprises 8 layers. In some embodiments, the compositions comprises 9 layers. In some embodiments, the compositions comprises 10 layers. In some embodiments, the composition comprises more than 1, 2, 3, 4, 5, 6, 7, 8, 9 or 10 layers. In some embodiments, the composition comprises more than 1 layer. In some embodiments, the composition comprises more than 2 layers. In some embodiments, the composition comprises more than 3 layers. In some embodiments, the composition comprises more than 4 layers. In some embodiments, the composition comprises more than 5 layers. In some embodiments, the composition comprises more than 6 layers. In some embodiments, the composition comprises more than 7 layers. In some embodiments, the composition comprises more than 8 layers. In some embodiments, the composition comprises more than 9 layers. In some embodiments, the composition comprises more than 10 layers.

In some embodiments the composition comprises 1 to about 20 layers. In some embodiments the composition comprises about 2 to about 20 layers. In some embodiments the composition comprises about 3 to about 20 layers. In some embodiments the composition comprises about 4 to about 20 layers. In some embodiments the composition comprises about 5 to about 20 layers. In some embodiments the composition comprises about 6 to about 20 layers. In some embodiments the composition comprises about 7 to about 20 layers. In some embodiments the composition comprises about 8 to about 20 layers. In some embodiments the composition comprises about 9 to about 20 layers. In some embodiments the composition comprises about 10 to about 20 layers. In some embodiments the composition comprises about 11 to about 20 layers. In some embodiments the composition comprises about 12 to about 20 layers. In some embodiments the composition comprises about 13 to about 20 layers. In some embodiments the composition comprises about 14 to about 20 layers. In some embodiments the composition comprises about 15 to about 20 layers. In some embodiments the composition comprises about 16 to about 20 layers. In some embodiments the composition comprises about 17 to about 20 layers. In some embodiments the composition comprises about 18 to about 20 layers. In some embodiments the composition comprises about 19 to about 20 layers.

In some embodiments the composition comprises 1 to about 20 layers. In some embodiments the composition comprises 1 to about 19 layers. In some embodiments the composition comprises 1 to about 18 layers. In some embodiments the composition comprises 1 to about 17 layers. In some embodiments the composition comprises 1 to about 16 layers. In some embodiments the composition comprises 1 to about 15 layers. In some embodiments the composition comprises 1 to about 14 layers. In some embodiments the composition comprises 1 to about 13 layers. In some embodiments the composition comprises 1 to about 12 layers. In some embodiments the composition comprises 1 to about 11 layers. In some embodiments the composition comprises 1 to about 10 layers. In some embodiments the composition comprises 1 to about 9 layers. In some embodiments the composition comprises 1 to about 8 layers. In some embodiments the composition comprises 1 to about 7 layers. In some embodiments the composition comprises 1 to about 6 layers. In some embodiments the composition comprises 1 to about 5 layers. In some embodiments the composition comprises 1 to about 4 layers. In some embodiments the composition comprises 1 to about 3 layers. In some embodiments the composition comprises 1 to about 2 layers.

In some embodiments, the method for preparing a multi-layered substantially cured or cured composition comprises preparing a first layer of a substantially cured or cured composition, wherein the preparing comprises mixing the curable compositions as provided for herein and exposing the curable composition to radiation to form a first layer of a substantially cured or cured composition. In some embodiments, the method comprises preparing a second layer of a substantially cured or cured composition on top of the first layer, the method comprising mixing a curable composition as provided for herein, wherein the curable composition comprises a photoinitiator, placing the mixture on top of the first layer, and exposing the mixture to radiation to form the second layer of a substantially cured or cured composition on top of the first layer. In some embodiments, the method comprises preparing a third layer of a substantially cured or cured composition on top of the second layer, the method comprising mixing a curable composition as provided for herein, wherein the curable composition comprises a photoinitiator, placing the mixture on top of the second layer, and exposing the mixture to radiation to form the third layer of a substantially cured or cured composition on top of the first layer. As provided for herein, this process can be repeated as many times as necessary for form a multi-layered composition.

As provided for herein, the curable compositions of the present disclosure may comprises radioisotopes or radioisotope precursors that are uniformly distributed throughout the composition, or may comprise a non-uniform distribution of the radioisotopes or radioisotope precursors. Accordingly, when creating a multi-layered substantially cured or cured composition via the methods provided for herein, each layer may optionally omit the radioisotope or radioisotope precursor composition in order to form a multi-layered substantially cured or cured composition wherein the radioisotope or radioisotope precursor of the multi-layered substantially cured or cured composition is spatially distributed such that a portion or portions of the multi-layered substantially cured or cured composition comprise a higher regional concentration of radioisotopes or radioisotope precursors than other regions of the multi-layered substantially cured or cured composition. Such a multi-layered substantially cured or cured composition would be useful, for example, to generate radiation dosimetry that conforms to a particular treatment area.

Accordingly, in some embodiments, the method for preparing a multi-layered substantially cured or cured composition comprises preparing a first layer of a substantially cured or cured composition, wherein the preparing comprises mixing the curable compositions as provided for herein and exposing the curable composition to radiation to form a first layer of a substantially cured or cured composition, preparing a second layer of a substantially cured or cured composition on top of the first layer, the preparing comprising mixing a curable composition comprising a photoinitiator as provided for herein, optionally wherein the curable composition does not comprise a radioisotope or radioisotope precursor, placing the mixture on top of the first layer, and exposing the mixture to radiation to form the second layer of a substantially cured or cured composition on top of the first layer; and optionally preparing a third layer of a substantially cured or cured composition on top of the second layer, the preparing comprising mixing a curable composition comprising a photoinitiator as provided for herein, optionally wherein the curable composition does not comprise a radioisotope or radioisotope precursor, placing the mixture on top of the second layer, and exposing the mixture to radiation to form the third layer of a substantially cured or cured composition on top of the first layer. As provided for herein, this process can be repeated as many times as necessary for form a multi-layered composition.

It is to be understood that the above referenced methods are exemplary and are not meant to be limiting in any way. For example, the preparing of the substantially cured or cured composition can comprise any necessary steps to initiate the process of curing or substantially curing the composition through the generation of free radicals in the third composition. In some embodiments, the free radicals are generated through a chemical reaction as provided for herein. In some embodiments the free radicals are generated when exposed to radiation as provided for herein. It is also to be understood that when generating a multi-layered substantially cured or cured composition that each layer may need not be exposed to an initiating catalyst (e.g. chemical reaction, radiation, etc.) to generate the free radicals. Without being bound to any particular theory, the free radicals generated by the third composition initiate the process of curing or substantially curing the composition. Each successive layer may be exposed to the initiating catalyst (e.g. chemical reaction, radiation, etc.) to generate free radicals, or the free radicals generated during the process of curing or substantially curing the preceding layer may themselves act as the initiating catalyst to cure or substantially cure the successive layer.

Accordingly, in some embodiments, the method for preparing a multi-layered substantially cured or cured composition comprises preparing a first layer of a substantially cured or cured composition, wherein the preparing comprises mixing the curable compositions as provided for herein and exposing the curable composition to radiation to form a first layer of a substantially cured or cured composition, preparing a second layer of a substantially cured or cured composition on top of the first layer, the preparing comprising mixing a curable composition as provided for herein, optionally wherein the curable composition does not comprise a radioisotope or radioisotope precursor, placing the mixture on top of the first layer, and optionally exposing the mixture to radiation, wherein the optional radiation or the free radicals generated during the preparation of the preceding layer initiate the process of forming the second layer of a substantially cured or cured composition on top of the first layer; and optionally preparing a third layer of a substantially cured or cured composition on top of the second layer, the preparing comprising mixing a curable composition as provided for herein, optionally wherein the curable composition does not comprise a radioisotope or radioisotope precursor, placing the mixture on top of the second layer, and optionally exposing the mixture to radiation, wherein the optional radiation or the free radicals generated during the preparation of the preceding layer initiate the process of forming the third layer of a substantially cured or cured composition on top of the first layer. As provided for herein, this process can be repeated as many times as necessary for form a multi-layered composition.

The compositions provided herein can also be prepared by an additive process, such as 3-D printing to form a composition that has a specific configuration or shape. The shape can be based on, for example, the area to be treated, such as the skin area or an internal cavity where the composition is going to be implanted.

Three-dimensional (3D or 3-D) printing is an additive printing process for making three-dimensional entities from digital models. 3D printing techniques are known as additive processes due to applications involving continuous layers of material.

3D printing technology is used in various industries for manufacturing and planning. For example, the automotive, aerospace, and consumer goods industries use 3D printing to make prototypes for parts and products. 3D printing is also used in the construction industry to print structural models. The use of 3D printing in private and government defenses is also growing rapidly.

Thus, 3-D printing can be used to create a construct (composition) according to an image of the area, cavity, surface, etc., of the subject that is to be treated. For example, conventional 3D printing allows objects to be created by depositing material on a flat manufacturing platform one layer at a time. After the deposition of the first layer, a second layer is deposited, i.e. on top of the first layer. The process is repeated as necessary to create a multi-layered solid article.

In some embodiments, a system is provided comprising a processor(s) that may be configured to prepare a model of the construct (i.e., the cured or substantially cured composition) that is to be produced by the system the 3-D printing system. The model construct may define the shape of the construct as well as the type(s) of material(s) to be included in the construct and the locations in the construct at which each type of material will be disposed. The shape of the construct may include a topographic shape, such as a topographic shape of a portion of the construct that is to interface subject or area of the subject that is to be treated. For example, a construct designed to merge with a portion of an subject with exposed skin or tissue may be made with compositions provided for herein.

In some embodiments, preparing the model may include scanning a region (e.g., skin, tissue, cavity and the like) of the subject that will receive the construct using a three-dimensional scanner. The scanner may be used, for example, to determine a topology of the region. The size of the construct or location may be identified through the scanning or in any other suitable way.

Alternatively or additionally, preparing the model may include downloading the model from a model repository or any other suitable source. The processor(s) may then generate the model of the construct based on the results.

In some embodiments, preparing of the model of the construct may be performed using commercially available software, which may include “off the shelf” scanning, modeling, and/or printing software. For example, common 3D model file formats may be used to create the tissue construct in common 3D modeling and printing software.

In some embodiments, the area to be radiated is determined by generating a dermoscopic image, which can then be translated into a 3-D image, which can be used to produce a construct through 3-D printing so that the area radiated is specifically radiated and the healthy tissue is not substantially affected by the radiation. Other systems can be used to create a 3-D image, which can then be used to generate the construct of the appropriate size or topography.

Accordingly, in some embodiments, methods of preparing a substantially cured or cured composition are provided. In some embodiments, the methods comprise mixing the curable composition as provided herein and printing the composition with a 3-D printer in a target area configuration, and exposing the composition to radiation to form a substantially cured or cured composition in the shape or topography of the target area configuration. In some embodiments, the third composition of the curable composition comprises a photoinitiator as provided for herein. In some embodiments, the target area configuration that is presented is prepared from a subject's area to be treated with the cured composition. In some embodiments, the target area is a skin surface that is to be treated. In some embodiments, the target area is a bone, tissue, cavity and the like of a subject. In some embodiments, the target area configuration is determined by imaging the subject's area to be treated with the cured composition to form the dimensions/boundaries of the target area configurations.

In some embodiments, methods of treating a subject with radiation are provided. In some embodiments, the methods comprise applying, implanting, contacting, and the like, the cured or substantially cured composition as provided for herein on, in, or with a target area of the subject. In some embodiments, the target area is a skin surface that is to be treated. In some embodiments, the target area is a bone, tissue, cavity and the like of a subject.

In some embodiments, methods of treating a subject with cancer are provided. In some embodiments, the methods comprise applying, implanting, contacting, and the like, the cured or substantially cured composition as provided for herein on, in, or with a target area of the subject. In some embodiments, the target area is a skin surface that is to be treated. In some embodiments, the target area is a bone, tissue, cavity and the like of a subject. In some embodiments, the cancer is skin cancer. In some embodiments, the skin cancer is malignant melanoma (MM), basal cell carcinoma (BCC) or squamous cell carcinomas (SCC). In some embodiments, the skin cancer is MM. The cured or substantially cured composition can be prepared according to a method as provided for herein.

### Enumerated Embodiments

In some embodiments, the following embodiments are provided:

1. A curable composition comprising:
a first composition comprising one or more monomers,
a second composition comprising one or more oligomers; and
a third composition capable of creating free radicals; and
a fourth composition comprising one or more radioisotopes.
   2. The curable composition of embodiment 1, wherein the one or more radioisotopes in the fourth composition is produced by activating one or more radioisotope precursors.
   3. The curable composition of embodiment 2, wherein the one or more radioisotope precursors is activated to produce one or more radioisotopes after the curable composition has been cured.
   4. The curable composition of any one of embodiments 1-3, wherein the one or more radioisotopes is present in an amount sufficient to be detected by an image processer.
   5. The curable composition of any one of embodiments 1-4, wherein the one or more radioisotopes is present in an amount sufficient to be detected and located.
   6. The curable composition of any one of embodiments 1-5, wherein the one or more radioisotopes is present in an amount sufficient to deliver a therapeutic dose of radiation to a mammal.
   7. The curable composition of embodiments 1-6, wherein the one or more radioisotopes comprise a diagnostic isotope, a therapeutic isotope, or a combination thereof.
   8. The curable composition of embodiment 7, wherein the diagnostic or therapeutic isotope is present in an amount sufficient to be detected and located.
   9. The curable composition of embodiment 7 or embodiment 8, wherein the diagnostic radioisotope is present in an amount sufficient to be detected by an image processor.
   10. The curable composition of any one of embodiments 7-9, wherein the therapeutic radioisotope is present in an amount sufficient to deliver a therapeutic dose of radiation to a mammal.
   11. The curable composition of any one of embodiments 1-10, wherein the one or more radioisotope is uniformly distributed throughout the composition.
   12. The curable composition of any one of embodiments 1-10, wherein the one or more radioisotope is not uniformly distributed throughout the composition.
   13. The curable composition of any one of embodiments 1-12, wherein the third composition creates free radicals through a chemical reaction.
   14. The curable composition of any one of embodiments 1-12, wherein the third composition creates free radicals when exposed to radiation.
   15. The curable composition of embodiment 14, wherein the radiation is thermal, ultraviolet, infrared, or the like.
   16. A curable composition comprising:
a first composition comprising one or more monomers,
a second composition comprising one or more oligomers; and
a third composition capable of creating free radicals when exposed to radiation (e.g., ultraviolet, infrared, and the like); and
a fourth composition comprising one or more radioisotope precursors.
   17. The curable composition of embodiment 16, wherein the one or more radioisotopes precursors in the fourth composition are activated to produce one or more radioisotopes.
   18. The curable composition of embodiment 17, wherein the one or more radioisotope precursors is activated to produce the one or more radioisotopes after the curable composition has been cured.
   19. The curable composition of any one of embodiments 16-18, wherein the one or more radioisotope precursors is present in an amount sufficient to produce an amount of one or more radioisotopes sufficient to be detected by an image processer.
   20. The curable composition of any one of embodiments 16-19, wherein the one or more radioisotope precursors is present in an amount sufficient to produce an amount of one or more radioisotopes sufficient to be detected and located.
   21. The curable composition of any one of embodiments 16-20, wherein the one or more radioisotope precursors is present in an amount sufficient to produce an amount of one or more radioisotopes sufficient to deliver a therapeutic dose of radiation to a mammal.
   22. The curable composition of any one of embodiments 16-21, wherein the one or more radioisotope precursors is activated to produce a diagnostic radioisotope, a therapeutic radioisotope, or a combination thereof.
   23. The curable composition of embodiment 22, wherein the one or more radioisotope precursors is present in an amount sufficient to produce a diagnostic radioisotope, a therapeutic radioisotope, or a combination thereof, in an amount sufficient to be detected and located.
   24. The curable composition of embodiment 22 or embodiment 23, wherein the one or more radioisotope precursors is present in an amount sufficient to produce a diagnostic radioisotope in an amount sufficient to be detected by an image processor.
   25. The composition of any one of embodiments 22-24, wherein the one or more radioisotope precursors is present in an amount sufficient to produce a therapeutic radioisotope in an amount sufficient to deliver a therapeutic dose of radiation to a mammal.
   26. The curable composition of any one of embodiments 16-25, wherein the one or more radioisotope precursors is uniformly distributed throughout the composition.
   27. The curable composition of any one of embodiments 16-25, wherein the one or more radioisotope precursors is not uniformly distributed throughout the composition.
   28. The curable composition of any one of embodiments 16-27, wherein the third composition creates free radicals through a chemical reaction.
   29. The curable composition of any one of embodiments 16-27, wherein the third composition creates free radicals when exposed to radiation.
   30. The curable composition of embodiment 29, wherein the radiation is thermal, ultraviolet,

31. The curable composition of any one of embodiments 1-30, wherein the curable composition is biocompatible.

32. The curable composition of any one of embodiments 1-31, wherein the curable composition is bioabsorbable.
33. The curable composition of any one of embodiments 1-32, wherein the curable composition further comprises one or more therapeutic agents.
34. The curable composition of embodiment 33, wherein the one or more therapeutic agents is an anticancer agent, an antiviral agent, an antibacterial agent, an antifungal agent, a pain management agent, an immunosuppressant agent, a radiation sensitizing agent, an anti-inflammatory agent, or a combination thereof.
35. The curable composition of any one of embodiments 1-34, wherein the first composition comprises a styrene, a N-vinylpyrrolidone, an acrylate, or a mixture thereof.
36. The curable composition of embodiment 35, wherein the styrene is 3-vinylphenylboronic acid, 1-vinylnaphthalene, 4-vinylbenzylamine, vinyltoluene monomer, 2,2′-(2-vinylanthracene-9,10-diylidene)bis(1,3-dithiole), 4-tert-butylstyrene, 2,3,4,5,6-pentafluorostyrene, 3-aminostyrene, 3-bromostyrene, 2-vinylanthraquinone, 4-fluoro-α-methylstyrene, 4-chlorostyrene, 2-chlorostyrene, chloromethylstyrene, 9-vinylanthracene, sodium p-styrenesulfonate hydrate, 4-methoxystyrene, 2-bromostyrene, 4-bromo-β,β-difluorostyrene, 3-fluorostyrene, 4-n-octylstyrene, 2,2′-(2-vinylanthracene-9,10-diylidene)dimalononitrile, divinylbenzene, 4-(chloromethyl)styrene, 2-vinylphenyl acetate, 3-chlorostyrene, vinylbenzyl cyanide, 3-(trifluoromethyl)styrene, 2,4,6-trimethylstyrene, trimethoxy(4-vinylphenyl)silane, 2-methylstyrene, 4-methylstyrene, 4-vinylbiphenyl, 4-isopropenyltoluene, 4-tert-butoxystyrene, 1-(1-ethoxyethoxy)-4-vinylbenzene, trimethyl(4-vinylphenyl)silane, 4-vinylphenyl acetate, α-methylstyrene, styrene, 4-aminostyrene, 4-fluorostyrene, 4-(trifluoromethyl)styrene, diphenyl(4-vinylphenyl)phosphine, 4-vinylbenzoic acid, 4-vinylphenylboronic acid, 4-nitrostyrene, 4-bromostyrene, 3-methylstyrene, or any combination thereof.
37. The curable composition of any one of embodiments 1-36, wherein the oligomer of the second composition comprises an epoxide, a urethanes, a polyethers, a polyester, or any combination thereof.
38. The curable composition of any one of embodiments 1-37, wherein the composition comprising one or more radioisotopes is in the form of a solution, colloid, microfibers, microspheres, nanotubes, or nanoparticles.
39. The curable composition of any one of embodiments 1-38, wherein the one or more radioisotopes is Pd-103, Pd-109, 1-125, 1-124, 1-123, I-131, P-32, Y-90, Ac-225, Cs-131, B-10, Ir-192, Sn-177m, Ho-166, Cu-64, Cu-67, Re-186, Re-188, Ga-67, Ga-68, In-111, Co-60, Cs-137, Lu-177, Yb-169, Er-169, Au-198, Sm-153, Am-241, Sr-89, Ra-223, Pb-212, Bi-213, Tc-99, At-211, Cf-252, or any combination thereof.
40. The curable composition of any one of embodiments 1-34, wherein the one or more radioisotopes is Pd-103, P-32, Y-90, or Sn-177m.
41. The curable composition of any one of any one of embodiments 2-40, wherein the one or more radioisotope precursors is Xe-124, S-32, Y-89, Sr-90, Th-229, Ir-191, Ir-193, Dy-164, Pd-102, Pd-104, Pd-108, B-10, P-31, Ni-64, Zn-67, Zn-68, Zn-70, Rh-103, Te-125, Ba-103, Cs-133, Te-124, Th-232, Sr-86, Er-168, Cd-111, Cd-112, W-186, Bi-209, Tm-169, Yb-176, Ra-226, or any combination thereof.
42. The curable composition of embodiment 40, wherein the one or more radioisotope precursors is selected from Pd-102, Pd-104, P-31, or Y-89.
43. The curable composition of any one of the preceding embodiments, wherein the monomer is a methylacrylate, the oligomer is an acrylate, and the one or more radioisotopes is Pd-103.
43A. The curable composition of embodiment 43, wherein the methylacrylate is 2-Hydroxyethyl Methacrylate (HEMA).
44. The curable composition of embodiments 43 or 43A, wherein the acrylate oligomer is urethane acrylate
45. The curable composition of any one of the preceding embodiments, wherein the monomer is a methylacrylate, the oligomer is an acrylate, and the one or more radioisotope precursors is Pd-102 or Pd-104.
46. The curable composition of embodiment 45, wherein the methylacrylate is 2-Hydroxyethyl Methacrylate (HEMA).
47. The curable composition of embodiments 45 or 46, wherein the acrylate oligomer is urethane acrylate
48. The curable composition of any one of embodiments 1-47, wherein the third composition comprises a photoinitiator, which can be a photochemical.
49. The curable composition of embodiment 48, wherein the photoinitiator is tert-Butyl peroxyneodecanoate, benzoyl peroxide, dicumyl peroxide, methyl ethyl ketone peroxide, lauryl peroxide cyclohexanone peroxide, t-butyl perbenzoate, t-butyl hyroperoxide t-butylbenzene hydroperoxide, cumene hydroperoxide, t-butyl peroctoate, azobis-isobutyronitrile, 2-t-butylazo-2-cyano-4-methylpentane, and 4-t-butylazo-4-cyano-valeric acid, or any combination thereof.
50. A substantially cured composition comprising a polymeric structure prepared from the curable composition of any one of the preceding embodiments.
51. A method of preparing a substantially cured composition, the method comprising mixing the curable composition of any one of embodiments 1-49 and exposing the curable composition to radiation to form a substantially cured composition.
52. The method of embodiment 51, wherein the method comprises:
preparing a first layer of a substantially cured composition, the preparing comprising mixing the curable composition of any one of embodiments 1-49 and exposing the curable composition to radiation to form a first layer substantially cured composition;
preparing a second layer of a substantially cured composition on top of the first layer, the preparing a second layer of a substantially cured composition on top of the first layer comprising mixing the curable composition of any one of embodiments 1-49 and a photoinitiator, placing the mixture on top of the first layer, and exposing the mixture to radiation to form a second layer of a substantially cured composition on top of the first layer; and
optionally preparing a third layer of a substantially cured composition on top of the second layer, then preparing a third layer of a substantially cured composition on top of the second layer comprising mixing the curable composition of any one of embodiments 1-49 and a photoinitiator, placing the mixture on top of the second layer, and exposing the mixture to radiation to form a third layer of a substantially cured composition on top of the second layer.
   53. A method of preparing a substantially cured composition, the method comprising mixing the curable composition of any one of embodiments 1-49 and printing the composition with a 3-D printer in a target area configuration and curing the composition by exposing the composition to radiation to form a substantially cured composition in the shape of the target area configuration.
   54. The method of embodiment 53, wherein the target area configuration that is presented is prepared from a subject's area to be treated with the cured composition.
   55. The method of embodiment 54, wherein the target area configuration is determined by imaging the subject's area to be treated with the cured composition to form the dimensions/boundaries of the target area configurations.
   56. A method of treating a subject with radiation, the method comprising applying, implanting, contacting, and the like, the cured composition prepared from of any one of embodiments 1-49.
   57. The method of embodiment 56, wherein the cured composition is prepared according to a method of any one of embodiments 51-55.
   58. The method of any one of embodiments 51-57, wherein the third composition of the curable composition comprises a photoinitiator.
   59. The method of embodiment 58, wherein the photoinitiator is tert-Butyl peroxyneodecanoate, benzoyl peroxide, dicumyl peroxide, methyl ethyl ketone peroxide, lauryl peroxide, cyclohexanone peroxide, t-butyl perbenzoate, tbutyl hydroperoxide, t-butylbenzene hydroperoxide, cumene hydroperoxide, t-butyl peroctoate, azobis-isobutyronitrile, 2-tbutylazo-2-cyano-4-methylpentane, and 4-t-butylazo-4-cyano-valeric acid, or any combination thereof.

The following examples are illustrative, but not limiting, of the compounds, compositions and methods described herein. Other suitable modifications and adaptations known to those skilled in the art are within the scope of the following embodiments.

### EXAMPLES

### Example 1. Preparation of a Composition Comprising a Radioactive Source

10 grams of PdCl₂ (The disc was produced using elemental palladium, for production of a disc for therapy an appropriate amount of Pd-103 mass will be substituted for an equivalent mass of elemental palladium) was loaded to an Ion exchange column containing 50 grams of resin and then stripped with concentrated ammonium hydroxide resulting in Pd Amine with a concentration of 6.363 g/L to form Pd(NH₃)₂Cl₂. DecorRom UV Resin—Clear was used and 2.33 grams of the DecorRom Resin and 600 uL of the Pd(NH₃)₂Cl₂ solution were mixed for approximately 2 minutes, the resulting solutions was a uniform yellow color. The concentration of the resulting solution was 1,276 ug Pd/gram of solution. The solution was then cured in a disc configuration with ANYCUBE Wash&Cure in layers. A 20 mm dia×1 mm deep silicon mold was used to form the disc. A total of 0.51 grams of the mixture from above was added and cured in three layers with each layer being cured for 4 minutes. The disc was found to be solid and stable 20 mm dia disc 1 mm tall with a weight is 0.51 grams; and the palladium contents of approximately 651 μg (calculated) and a Pd-103 capacity of 46 Curies (calculated). The cured composition was uniform light orange in color.

### Example 2. 3-D Printing of Composition Comprising a Radioactive Source

In this example, the 3-D printed disc was produced using elemental palladium, for production of a disc for therapy an appropriate amount of Pd-103 mass will be substituted for an equivalent mass of elemental palladium. 10 grams of PdCl₂ was loaded to an Ion exchange column containing 50 grams of resin and then stripped with concentrated ammonium hydroxide resulting in Pd Amine with a concentration of 6.363 g/L of Pd(NH₃)₂Cl₂. The disc was printed using a Weistek 3D Printer Resin, a 405 nm LCD UV Curing Photopolymer Resin. Briefly, 45 mL of the Weistek 3D Print Resin and 5 mL of the Pd(NH₃)₂Cl₂ solution were mixed for approximately 8 minutes, the resulting solutions was a uniform milky white color. The concentration of the resulting solution was 470 pg Pd/gram of solution. The 3D-Print was performed ANYCUBE PHOTON S 405 nm UV Wavelength printer by depositing the solution above in a disc configuration. The deposited disc solution was washed with ANYCUBE Wash&Cure and then with isopropyl alcohol and then cured with ANYCUBE Wash&Cure. The disc that was produced was solid and stable 20.2 mm dia disc 1.6 mm tall with a weight of 0.59 grams. The palladium contents were approximately 278 ug (calculated) with a Pd-103 capacity of 20 Curies (calculated) and had uniform milky white color.

### Example 3. Preparation of a Composition Comprising a Radioactive Source

In this example, the disc was produced using glass microspheres containing elemental yttrium, for production of a disc for therapy an appropriate amount of yttrium will be activated to Y-90. 1.24 g of DecorRom UV Resin—Clear and 250 mg of glass microspheres containing approximately 79 mg of Y-89 were mixed for approximately 2 minutes, the resulting solution was colorless and opaque. The microspheres can be glass, or other materials, and can be formulated to contain a number of therapeutic isotopes, including, but not limited to, Y-90, P-32, and Ho-166. A 20 mm diameter by 1 mm deep silicon mold was used to form a disc. A total of 0.50 g of the solution from above was added as a single layer and cured for 4 minutes with ANYCUBE Wash&Cure. The disc was found to be solid and stable (20 mm diameter disc, 1 mm tall with a weight of 0.51 g) with a Y-89 content of approximately 29 mg (calculated) and a Y-90 capacity of 160 mCi (calculated). The cured composition was uniformly colorless and opaque.

### Example 4. Preparation of a Composition Comprising a Radioactive Source

In this example, the disc was produced using a tin colloid containing stable elemental tin, for production of a disc for therapy an appropriate amount of Sn-117m will be substituted for stable tin. 1.09 grams of DecorRom UV Resin—Clear and 410 mg of tin colloid containing approximately 1.22 mg of natural tin were mixed for approximately 2 minutes, the resulting solution was colorless and opaque. A 20 mm diameter by 1 mm deep silicon mold was used to form a disc. A total of 0.50 g of the solution from above was added as a single layer and cured for 4 minutes with ANYCUBE Wash&Cure. The disc was found to be solid and stable (20 mm diameter disc, 1 mm tall with a weight of 0.51 g) with a tin content of approximately 1.22 mg (calculated) and an Sn-117m capacity of approximately 96 Ci (calculated). The cured composition was uniformly colorless and opaque.

### Example 5. Preparation of a Composition with Spatially Distributed Radioisotopes

Generation via multi-layered composition. A silicon mold is used as in the previous examples to generate a first disc layer comprising an effective amount of a radioisotope or radioisotope precursor as provided for herein. Successive layers are added on top of the first layer, with successive layers containing less or no radioisotope or radioisotope precursor. The resulting multi-layered disc comprises a cured or substantially cured composition having spatially distributed radioisotopes or radioisotope precursors.

Generation via 3-D printing. A target tissue area is imaged to create a 3-D map of the target area. A mold of the desired shape is 3-D printed and layers of curable compositions as provided for herein are added to the mold. Each layer may have the same or a different concentration of radioisotope or radioisotope precursor. Wherein each layer has different concentrations of radioisotope or radioisotope precursor, a multilayered cured or substantially cured composition is generated having spatially distributed radioisotopes or radioisotope precursors.

Alternatively, the desired concentration of radioisotopes or radioisotope precursors are added to the 3-D printing resin, and the 3-D printer is used to print each layer of the cured or substantially cured composition. Multiple compositions are provided having independent radioisotope or radioisotope precursor concentrations such that the 3-D printer prints a cured or substantially cured composition having spatially distributed radioisotopes or radioisotope precursors.

### Example 6

A patient presents to a doctor with melanoma. The melanoma is imaged to create a 3-D map of the target tissue area. The 3-D map is used to create a cured composition comprising a radioactive source according to as provided for herein and as illustrated in Examples 1-4. The cured composition comprising the radioactive source is applied to the patient's melanoma and is treated with radiation from the composition without substantially radiating healthy tissue surrounding the melanoma.

The present examples demonstrate that a curable composition comprising a radioactive source can be made in a specific configuration and used in therapy as provided for herein.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

What is claimed is:

1. A curable composition comprising:
a first composition comprising one or more monomers,
a second composition comprising one or more oligomers; and
a third composition capable of creating free radicals; and
a fourth composition comprising one or more radioisotopes.

2. The curable composition of claim 1, wherein the one or more radioisotopes in the fourth composition is produced by activating one or more radioisotope precursors.

3. The curable composition of claim 2, wherein the one or more radioisotope precursors is activated to produce one or more radioisotopes after the curable composition has been cured.

4. The curable composition of claim 1, wherein the one or more radioisotopes comprise a diagnostic isotope and a therapeutic isotope.

5. The curable composition of claim 4, wherein the diagnostic radioisotope is present in an amount sufficient to be detected and located.

6. The curable composition of claim 4, wherein the therapeutic radioisotope is present in an amount sufficient to deliver a therapeutic dose of radiation to a mammal.

7. The curable composition of claim 1, wherein the one or more radioisotopes is not uniformly distributed throughout the composition.

8. The curable composition of claim 1, wherein the third composition creates free radicals through a chemical reaction or when exposed to radiation, wherein the radiation is thermal, ultraviolet, or infrared radiation.

9. The curable composition of claim 1, wherein the curable composition is biocompatible.

10. The curable composition of claim 1, wherein the curable composition is bioabsorbable.

11. The curable composition of claim 1, wherein the curable composition further comprises one or more therapeutic agents, wherein the one or more therapeutic agents is an anticancer agent, an antiviral agent, an antibacterial agent, an antifungal agent, a pain management agent, an immunosuppressant agent, a radiation sensitizing agent, an anti-inflammatory agent, or a combination thereof.

12. The curable composition of claim 1, wherein the first composition comprises a styrene, a N-vinylpyrrolidone, an acrylate, or a mixture thereof.

13. The curable composition of claim 12, wherein the styrene is 3-vinylphenylboronic acid, 1-vinylnaphthalene, 4-vinylbenzylamine, vinyltoluene monomer, 2,2′-(2-vinylanthracene-9,10-diylidene)bis(1,3-dithiole), 4-tert-butylstyrene, 2,3,4,5,6-pentafluorostyrene, 3-aminostyrene, 3-bromostyrene, 2-vinylanthraquinone, 4-fluoro-α-methylstyrene, 4-chlorostyrene, 2-chlorostyrene, chloromethylstyrene, 9-vinylanthracene, sodium p-styrenesulfonate hydrate, 4-methoxystyrene, 2-bromostyrene, 4-bromo-β,β-difluorostyrene, 3-fluorostyrene, 4-n-octylstyrene, 2,2′-(2-vinylanthracene-9,10-diylidene)dimalononitrile, divinylbenzene, 4-(chloromethyl)styrene, 2-vinylphenyl acetate, 3-chlorostyrene, vinylbenzyl cyanide, 3-(trifluoromethyl)styrene, 2,4,6-trimethylstyrene, trimethoxy(4-vinylphenyl)silane, 2-methylstyrene, 4-methylstyrene, 4-vinylbiphenyl, 4-isopropenyltoluene, 4-tert-butoxystyrene, 1-(1-ethoxyethoxy)-4-vinylbenzene, trimethyl(4-vinylphenyl)silane, 4-vinylphenyl acetate, α-methylstyrene, styrene, 4-aminostyrene, 4-fluorostyrene, 4-(trifluoromethyl)styrene, diphenyl(4-vinylphenyl)phosphine, 4-vinylbenzoic acid, 4-vinylphenylboronic acid, 4-nitrostyrene, 4-bromostyrene, 3-methylstyrene, or any combination thereof.

14. The curable composition of claim 1, wherein the oligomer of the second composition comprises an epoxide, a urethanes, a polyethers, a polyester, or any combination thereof.

15. The curable composition of claim 1, wherein the composition comprising one or more radioisotopes is in the form of a solution, colloid, microfibers, microspheres, nanotubes, or nanoparticles.

16. The curable composition of claim 1, wherein the one or more radioisotopes is Pd-103, Pd-109, 1-125, 1-124, 1-123, 1-131, P-32, Y-90, Ac-225, Cs-131, B-10, Ir-192, Sn-177m, Ho-166, Cu-64, Cu-67, Re-186, Re-188, Ga-67, Ga-68, In-111, Co-60, Cs-137, Lu-177, Yb-169, Er-169, Au-198, Sm-153, Am-241, Sr-89, Ra-223, Pb-212, Bi-213, Tc-99, At-211, Cf-252, or any combination thereof.

17. The curable composition of claim 2, wherein the radioisotope precursor is Xe-124, S-32, Y-89, Sr-90, Th-229, Ir-191, Ir-193, Dy-164, Pd-102, Pd-104, Pd-108, B-10, P-31, Ni-64, Zn-67, Zn-68, Zn-70, Rh-103, Te-125, Te-124, Ba-130, Cs-133, Th-232, Sr-86, Er-168, Cd-111, Cd-112, W-186, Bi-209, Tm-169, Yb-176, Ra-226, or any combination thereof.

18. The curable composition of claim 1, wherein the monomer is a methylacrylate, the oligomer is an acrylate, and the one or more radioisotopes is Pd-103.

19. The curable composition of claim 18, wherein the methylacrylate is 2-Hydroxyethyl Methacrylate (HEMA) and the acrylate oligomer is urethane acrylate.

20. The curable composition of claim 1, wherein the third composition comprises a photoinitiator, wherein the photoinitiator is tert-Butyl peroxyneodecanoate, benzoyl peroxide, dicumyl peroxide, methyl ethyl ketone peroxide, lauryl peroxide cyclohexanone peroxide, t-butyl perbenzoate, t-butyl hyroperoxide t-butylbenzene hydroperoxide, cumene hydroperoxide, t-butyl peroctoate, azobis-isobutyronitrile, 2-t-butylazo-2-cyano-4-methylpentane, and 4-t-butylazo-4-cyano-valeric acid, or any combination thereof.

21. A substantially cured composition comprising a polymeric structure prepared from the curable composition of claim 1.

22. A method of preparing a substantially cured composition, the method comprising mixing the curable composition of claim 1 and exposing the curable composition to radiation to form a substantially cured composition.

23. The method of claim 22, wherein the method comprises:
preparing a first layer of a substantially cured composition, the preparing comprising mixing the curable composition of claim 1 and exposing the curable composition to radiation to form a first layer substantially cured composition;
preparing a second layer of a substantially cured composition on top of the first layer, the preparing a second layer of a substantially cured composition on top of the first layer comprising mixing the curable composition of claim 1 and a photoinitiator, placing the mixture on top of the first layer, and exposing the mixture to radiation to form a second layer of a substantially cured composition on top of the first layer; and
optionally preparing a third layer of a substantially cured composition on top of the second layer, then preparing a third layer of a substantially cured composition on top of the second layer comprising mixing the curable composition of claim 1 and a photoinitiator, placing the mixture on top of the second layer, and exposing the mixture to radiation to form a third layer of a substantially cured composition on top of the second layer.
